(19) United States
(12) Patent Application Publication
(10) Pub. No.: US 2019/0150795 A1
(43) Pub. Date: May 23, 2019

(54) SYSTEM AND USE METHOD FOR DETECTING AND CORRECTING INCORRECT POSTURE

(71) Applicant: Bin Lu, Saratoga, CA (US)

(72) Inventor: Bin Lu, Saratoga, CA (US)

(21) Appl. No.: 15/821,748

(22) Filed: Nov. 22, 2017

(51) Int. Cl. A61B 5/11; A61B 5/00; G09B 1/06; G09B 19/00

(52) U.S. Cl. CPC A61B 5/1116; A61B 5/6802; A61B 5/7455; G09B 19/00; A61B 2560/0214; G09B 1/06

(57) ABSTRACT

The system is a closed-loop feedback mechanism that helps a user correct improper posture. The system consists of two or more system enclosures on opposite ends of a loop formed by connecting the enclosures with a necklace portion and multi-conductor cable of essentially equal length. Worn around the neck, one system enclosure is centered on the upper chest and the other enclosure is centered on the upper back. The two enclosures measure the angle of head back and neck to upper body and detect when a user is slouching. When so detected for some predetermined period of time, a transducer in one enclosure vibrates silently to alert the user that he or she is slouching. The vibratory alert ends when a user corrects his or her posture.

### TECHNICAL FIELD

This is a system related to human posture detection and correction.

### BACKGROUND OF THE INVENTION

When a human being stands, walks or sits with best posture, stress on the skeletal and muscular systems are reduced. Slouching, where the head and upper body rotate slightly forward and the head's mass is putting both compressional and lateral stress on the upper spinal column, is a common posture issue that can degrade one's health.

Devices have been invented that attach to a person's body or clothing and are used to detect angular displacement from the vertical (as defined by a plumb line). When some predetermined value of displacement is measured, these devices vibrate to let the user know that the user is apparently using incorrect posture. Once the person straightens up, and the angular displacement is reduced below the predetermined level, the vibration or other signaling ceases. Thus, it is a form of feedback that is believed to help someone overcome incorrect posture.

One issue with these contemporary devices is they use gravitational vertical as the baseline for measuring angular displacement. That, of course, assumes that the person using the device is either standing erect or sitting erect. If someone is reclining, the device will detect an incorrect posture event even though the person's head and spinal column may be in proper alignment.

A device that could measure the difference in angle of upper torso and head/neck with respect to some reference could provide a way to determine incorrect posture, or slouching, without relying on a gravitational vertical baseline.

### BRIEF SUMMARY OF THE INVENTION

The invention disclosed is a system and use method whereby two separate detectors measure some angular displacement relative to a reference, and therefore, to one another. Thus, alignment of upper torso and head/neck is not dependent upon a gravitational vertical baseline. Someone sitting in a chair, for example, where the chair is slightly reclining, would still receive accurate posture feedback because the two separate detection enclosures would essentially find no change in postural angular displacement though the user is slightly reclined. The same detection scheme could detect when a person is essentially horizontal, as when laying down, and turn off both detectors, thus extending battery life.

The two separate detection enclosures are joined by a necklace portion and interface cable portion such that they are essentially diametrically opposite one another, with one enclosure behind the head and resting on the upper back, and the other below the head and resting on the chest area. Their relative positions are stabilized by the weight of a battery located in the enclosure resting on the chest area. That weight loads the necklace and interface cable so as to keep the two enclosures in proper and stable positions. However, the weight is small enough so that it does not add any measurable discomfort to the user.

Initial calibration of each user's system is done while standing, erect, and initializing a calibration function. Once calibrated, any angular displacement due to body shape differences are factored into any baseline reference value. Subsequent measurements use that baseline reference value against which each enclosure's angular displacement measurement is determined. This will normalize the readings so that reclining will not lead to inaccurate postural detection.

### BRIEF DESCRIPTIONS OF THE DRAWINGS

FIG. 1 depicts the enclosures comprising the system in one embodiment.

FIG. 2 depicts external system components.

FIG. 3 illustrates how the system is worn by a user.

FIG. 4 depicts the enclosures comprising the system in a second embodiment.

FIG. 5 depicts the enclosures comprising the system in a third embodiment.

FIG. 6 depicts the enclosures comprising the system with an added component.

FIG. 7 depicts the external system components including that of FIG. 6.

FIG. 8 illustrates how the system of FIGS. 6 and 7 is worn by a user.

### DETAILED DESCRIPTION OF THE INVENTION

Proper posture can play an important role in maintaining optimal skeletal and muscular health. When standing erect, with proper posture, the upper body mass is exerting essentially vertical compressive force on the skeletal system. Slouching forward, however, adds some lateral stress on the spine and can result not only in poor posture but also muscular and joint pain.

Most people are usually unaware when they deviate from a proper postural position while standing, walking or sitting. The system disclosed herein provides a feedback mechanism that silently vibrates to alert a user when he or she is slouching. So long as the user remains slouching for a predetermined period of time, the feedback signal is present. Once the user corrects the postural deviation, the signal stops. When a user is thus alerted to postural deviation, most typically make an effort to stop the feedback signal. This results in less time in an improper postural position and can result in better muscular/skeletal alignment.

Because of the differences in each person's body shape, a system meant to detect and alert a user about postural deviation must take these differences into account. This can be done by calibrating the system at first use by standing erect in proper postural position, and allowing the system to establish a baseline reference for proper posture.

Once the baseline calibration has been done, while a user is sitting, standing or walking erect, the system frequently measures the orientation of head/neck and chest against those same measurements comprising the baseline value. When the system detects that someone's head/neck orientation with regard to chest has changed beyond some predetermined value for a predetermined period of time, the system sends an alert signal to a transducer that silently vibrates and alerts the user. When the user responds to the vibration by adjusting head/neck position with regard to chest, the system will detect that the user is once again within proper postural value limits and cease the alert signal and vibration. It is a classic closed-loop feedback scenario. The transducer may also initiate a signal via Bluetooth LE to a nearby smartphone where day and time are recorded in a user history for later review.

The system comprises a first enclosure and a second enclosure wherein each enclosure contains one or more angular sensors, such as a 3D accelerometer, operative to provide positional input. It is the difference in angular displacement between the two enclosures, when standing, sitting or walking erect, that comprises the baseline value. Subsequent comparisons of the accelerometer readings, with reference to a predetermined value, are used by the system to determine whether a person is slouching. When the system detects a slouching condition, it sends an alert signal to a transducer in one of the enclosures that begins a silent vibration that is readily sensed by the user.

The system's two enclosures are positioned behind the head/neck and on the upper chest. They are held in place by a necklace portion on one side, and a multi-conductor cable on the other side such that the enclosures, necklace portion, and cable portion form a physical closed loop. The length of the necklace portion is essentially the same as the multi-conductor cable thereby keeping both enclosures centered and in a stable position, one in front, and one in back.

With the enclosure that is positioned on the upper chest containing the battery, its weight will be sufficient to align the two enclosures into a proper and stable position on the user's body. Both enclosures are relatively small and light in weight, as are the necklace portion and multi-conductor cable. Thus, the additional battery weight of the one enclosure is sufficiently larger than that of the other to provide positional stabilization.

The multi-conductor cable may have a connector on at least one end that mates with a connector on one of the two enclosures such that it can be detached from the enclosure to allow placing the system around the user's neck, connecting the cable end, and forming a closed physical loop. Alternatively, the necklace portion and multi-conductor cable may be affixed to both enclosures such that the closed loop is positioned above the head and passed downward into position.

A replaceable button-cell battery or rechargeable battery in one enclosure provides the DC power for the electronic components in each enclosure. Power is routed to the enclosure having no battery by the multi-conductor cable.

A momentary switch on the enclosure that sits on the upper chest may be closed when the user is standing erect to thus initiate a calibration operation and establish a baseline value. Alternative methods of initiating a calibration sequence may also be used. Once calibrated, the system continues to monitor the 3D accelerometer readings to determine if a user is slouching or not. When slouching is detected for a predetermined time period, the alert signal is initiated and may be maintained until slouching is no longer detected.

The system may include a process whereby the 3D accelerometers can be detected to determine if a person is essentially horizontal—that is, laying down. It can be programmed such that when horizontal orientation is detected, the system puts itself into a low-energy state whereby infrequent polling of 3D accelerometer readings is done until the system determines that the user is no longer supine. This could extend battery life.

An alternative approach might use a position-sensitive switch which remains closed while someone is standing, walking or sitting but opens to turn off the power when someone is laying down. This, too, could extend battery life. To ensure that either enclosure containing a position-sensitive switch is always properly oriented, the necklace portion and multi-conductor cable may be attached to the enclosures near the portion that is to remain closest to the neck or chest and allowing component weight to keep the enclosures in that proper position even when user lays down on either side. The position-sensitive switch is always in an off state in such cases.

Systems that measure a user's position relative to a gravitational baseline are known in the art. These typically comprise a single device that is worn on clothing and positioned on the upper chest. However, such devices could detect an improper posture when a user is reclining in a chair though the head/neck and chest alignment is still proper. The system herein disclosed avoids false positives by using two or more 3D accelerometers. The baseline, in this case, is established by comparing the two readings during calibration, and is not slaved to a fixed gravitational vertical line. Thus, the detection of improper posture using the system disclosed is much less subject to error caused by a reclining user situation.

One use method embodiment comprises placing the system around a user's neck, attaching the free end of the multi-conductor cable to the connector on the enclosure to which the cable is not yet connected, then orienting the enclosures such that the one without the battery is located essentially in the middle of the area behind the head and neck on the upper back, and the enclosure containing the battery is located essentially in the middle of the upper chest.

Once so positioned, the user stands erect and initiates a calibration sequence. Once calibrated, the system continues to monitor 3D accelerometer readings, comparing them to baseline value, and generating an alert signal when readings deviate beyond some predetermined value. Once the readings are again within the proper posture range, the system ceases generating the alert signal.

The sizes and shapes of the enclosures may vary. The material used for the necklace portion may be metallic, non-metallic, and should be light in weight yet reasonably strong. The multi-conductor cable should be flexible and use wire gauge able to provide sufficiently low resistance with light weight. Current carrying capacity can be quite low since the system uses very little energy, even when operating the transducer.

A microcontroller chip resides in one enclosure and accepts output from the two or more 3D accelerometers while comparing their readings to programmed values. The program determines when the readings constitute a deviation in postural position and then generates the alert signal. During use, when a user breathes, the angle detected by the 3D accelerometer in one enclosure changes with the rise and fall of the upper chest. The program takes account of this in determining the angle. However, that angular change is directly related to a user's breathing rate. As such, that reading can also be captured and used to provide feedback related to a user's breathing rate. This can be an indicator if the user is under stress.

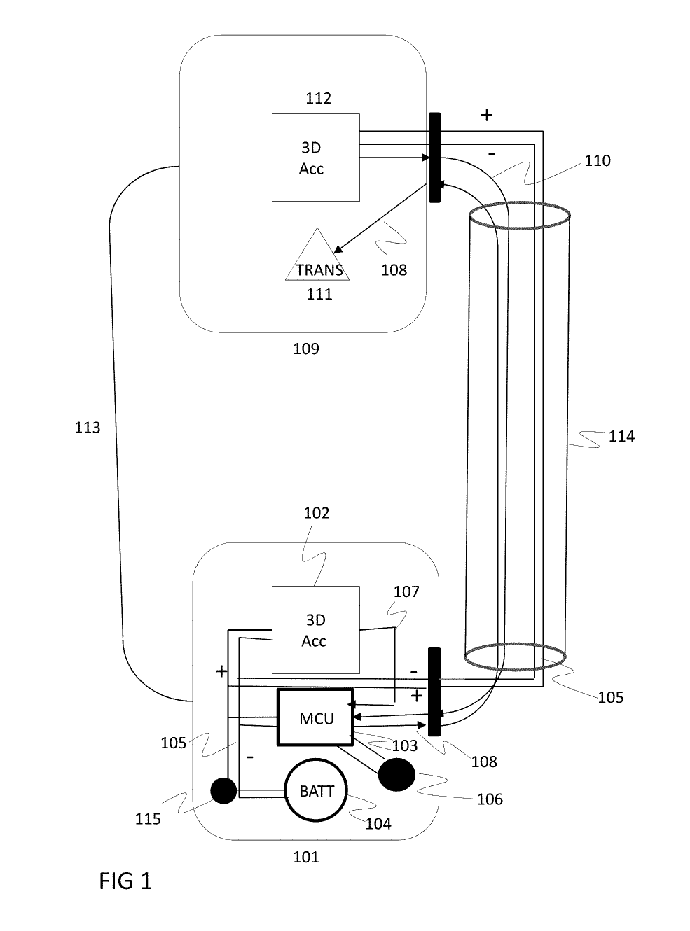

In FIG. 1, a first enclosure (101) comprises a 3D accelerometer (102), a microcontroller (103), and a battery (104). Power is distributed to the microcontroller and 3D accelerometer over conductors (105). A second enclosure (109) comprises a second 3D accelerometer (112), and a vibrating transducer (111). The vibrating transducer could alternatively be located in a first enclosure (101). The power provided by the battery is also routed to the second enclosure over the same conducting path (105). The output of 102 is routed to the microcontroller over conducting path 107. The output of 112 is routed to the microcontroller through cable 114 via conducting path 110. When an alert is generated, the alert signal is routed to the transducer (111) over conducting path 108. The necklace portion is 113, and the multi-conductor cable portion is 114. In this embodiment, calibration is initiated by momentary closure of switch 106. The position-sensitive switch, 115, may be included so as to turn off power when the user is lying down.

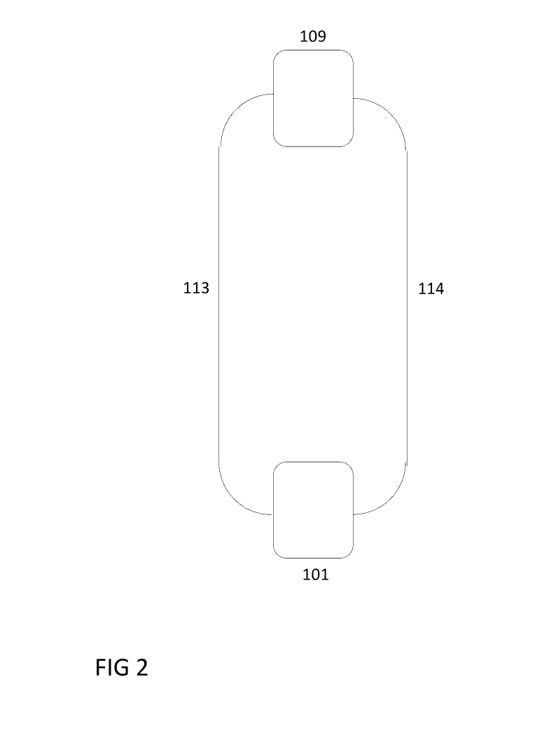

FIG. 2 shows the two enclosures, the necklace portion, and multi-conductor cable portion forming a physical closed loop.

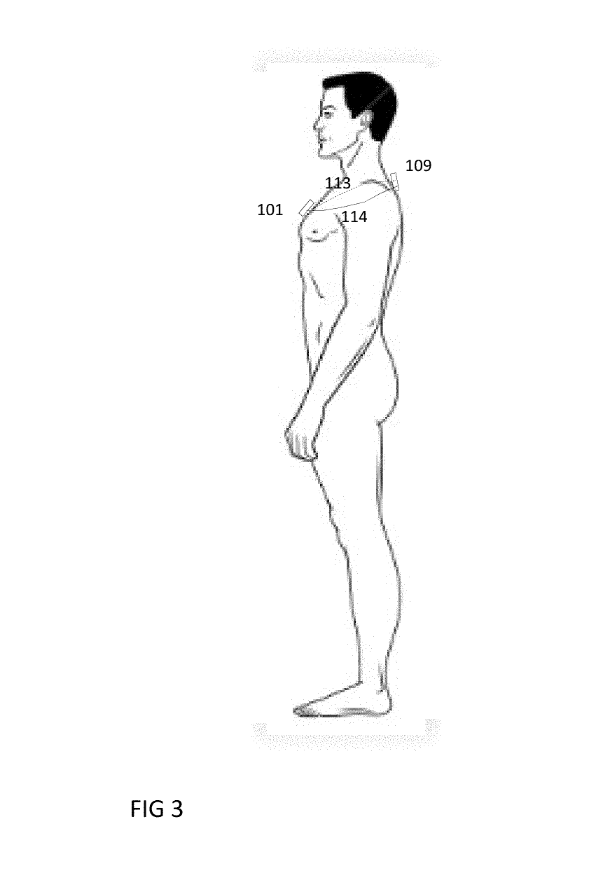

FIG. 3 shows the enclosure 109 located behind the head and neck on the upper back, enclosure 101 located in front on the upper chest. The multi-conductor cable 114 is oriented on the user's left side, and the necklace portion (113) is oriented on the user's right side.

The orientation of the necklace portion and multi-conductor cable is arbitrary. Their positions can be reversed. The position of components within each enclosure is not crucial but the battery should be located so as to place essentially the same force on both the necklace portion and multi-conductor cable portion. FIG. 1 shows the transducer in one enclosure and the battery and microcontroller in a second enclosure. It should be noted that the microcontroller can be located in the same enclosure as the transducer.

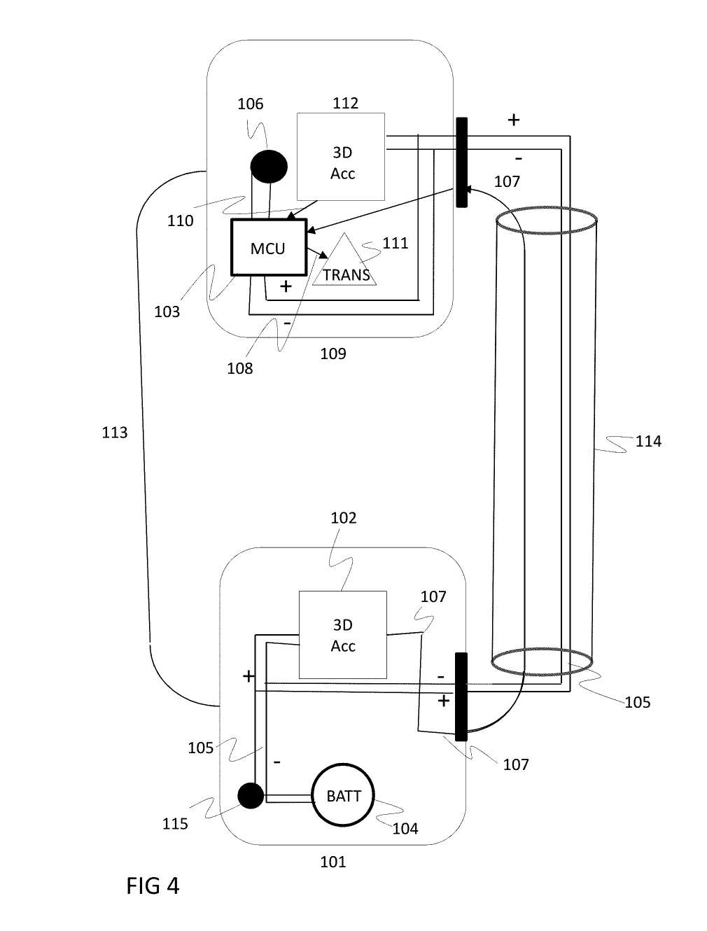

Thus, in FIG. 4, the microcontroller (103), and calibration switch (106) are located in enclosure 109. The alert signal 108 goes directly to the transducer (111) and its conducting path is contained with enclosure 109. That is, it does not require a conductor in the multi-conductor cable portion (114).

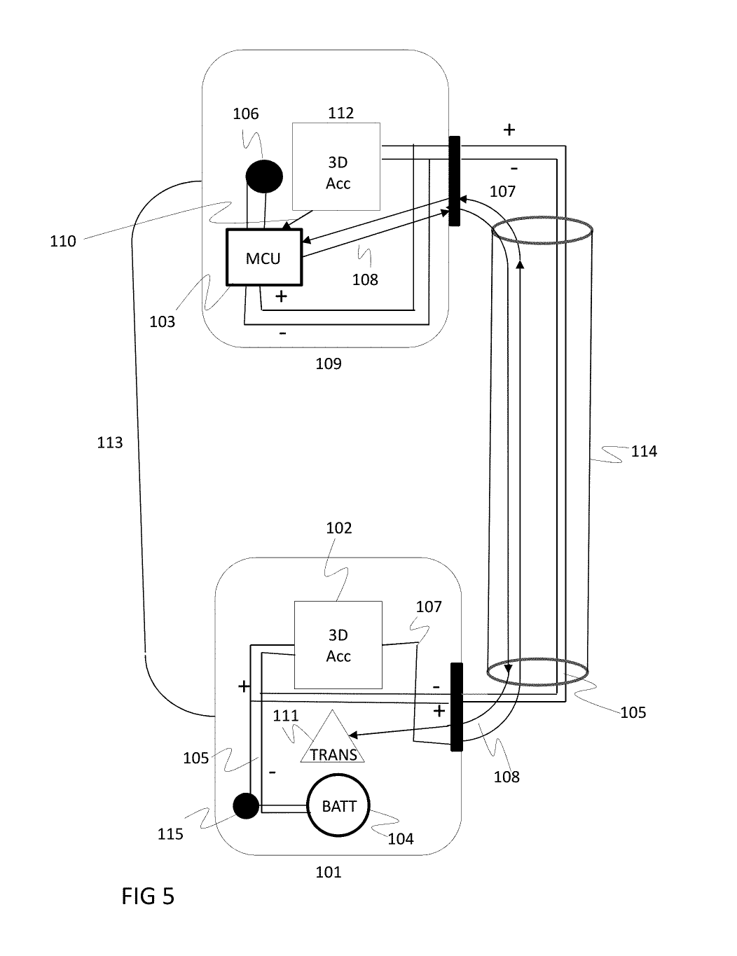

In another embodiment, FIG. 5, the microcontroller is located in enclosure 109 whereas the transducer is located in enclosure 101. The alert signal 108 from the microcontroller does now require a conductor in the multi-conductor cable portion in order to convey the signal from enclosure 109 to enclosure 101 and the transducer (111).

Although not shown, the transducer and microcontroller may also be located in enclosure 101.

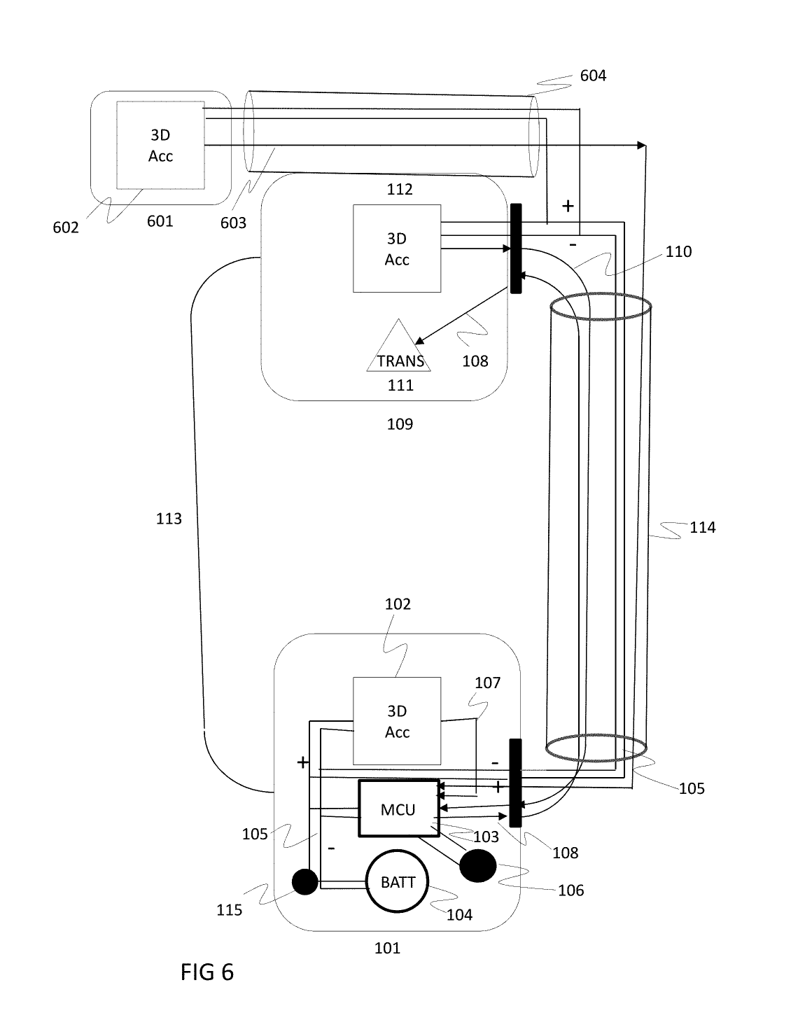

As shown in FIG. 6, an additional 3D accelerometer may be enclosed in a third enclosure (601) and DC power and output signals are conveyed by a second multi-conductor cable portion (604). The output conducting path is 603 which conveys output signal to a microcontroller located in either enclosure 101 or enclosure 109. This can provide more precise posture information because a person may maintain good posture relative to head/neck and upper back, but be slouching due to bending of the lower back.

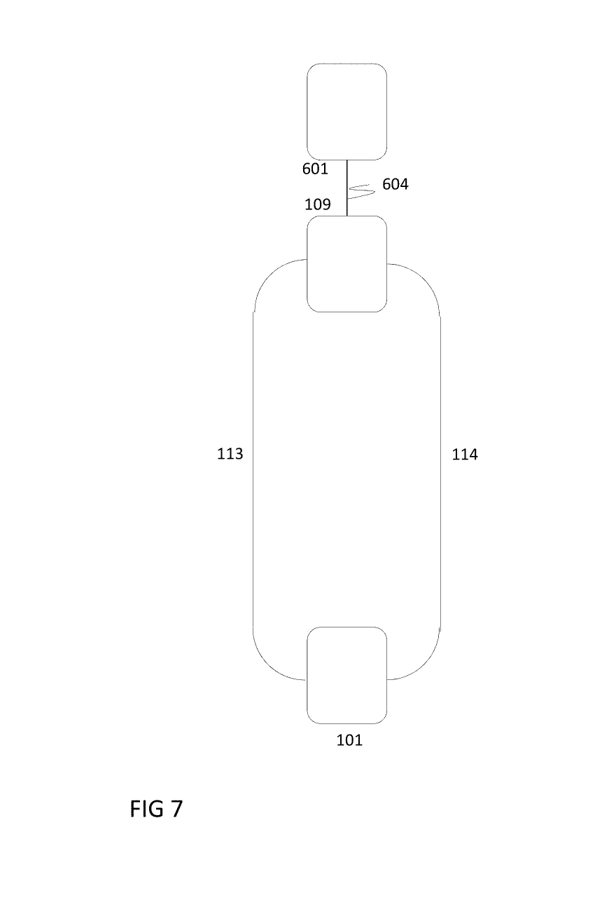

FIG. 7 shows the system with the addition of the enclosure 601. The length of multi-conductor 604 is such that it allows the enclosure 601 to extend further down the user's back than enclosure 109. The additional 3D accelerometer may provide additional precision to the angular measurement of head/neck to lower back.

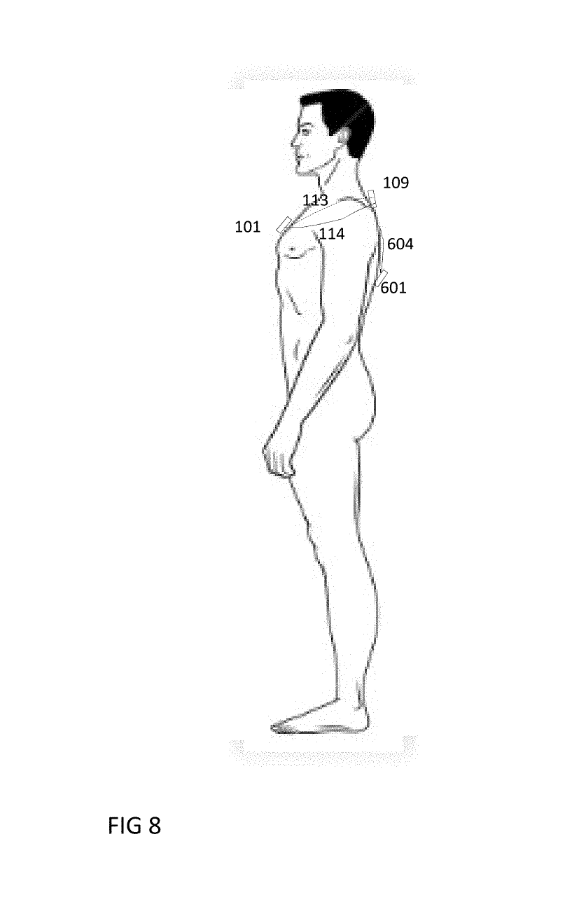

FIG. 8 shows the position of the three enclosures, necklace portion, and multi-conductor cable portions on a user's body.

The microcontroller can be both low cost and small. Processing speeds are not critical. The 3D accelerometers can be both low cost and small. Resolution can be degrees or lower. The transducer can be both low cost and small. It should be mounted so as to efficiently transfer its vibration force to the enclosure of whichever enclosure it is located.

What is claimed is:

1. A system comprising:
two or more 3D accelerometers;
a battery;
a microcontroller;
a silent vibratory transducer;
two or more enclosures;
a necklace portion;
one or more multi-conductor cable portions;
a position-sensitive switch;
a momentary switch; and
one or more embedded programs.

2. A claim as in claim 1 further comprising:
a first of said two or more enclosures to be positioned on a user's upper chest area; and
a second of said two or more enclosures to be position on said user's upper back area.

3. A claim as in claim 1 further comprising:
a first of said two or more 3D accelerometers enclosed in said first of said two or more enclosures;
said battery enclosed in said first of said two or more enclosures;
said microcontroller enclosed in said first of said two or more enclosures;
said silent vibratory transducer enclosed in said first of said two or more enclosures a second of said two or more 3D accelerometers enclosed in said second of said two or more enclosures;
said necklace portion attached at a first end to said first of two or more enclosures;
said necklace portion attached at a second end to said second of two or more enclosures;
said first of said one or more multi-conductor cable portions attached at one end to said first to two or more enclosures;
said first of said one or more multi-conductor cable portions attached at a second end to said second of two more enclosures;
said first of said one or more multi-conductor cable portions operative to convey electrical energy to components from said first of said two or more enclosures to said second of said two or more enclosures;
said position-sensitive switch enclosed in said first of two or more enclosures;
said momentary switch enclosed in said first of two or more enclosures; and
said one or more embedded programs stored in said microcontroller.

4. A claim as in claim 1 further comprising:
said first of said two or more 3D accelerometers enclosed in a first of said two or more enclosures;
said battery enclosed in said first of said two or more enclosures;
said microcontroller enclosed in said second of said two or more enclosures;
said silent vibratory transducer enclosed in said second of said two or more enclosures;
a second of said two or more 3D accelerometers enclosed in a second of said two or more enclosures;
said necklace portion attached at a first end to said first of two or more enclosures;
said necklace portion attached at a second end to said second of two or more enclosures;
said first of said one or more multi-conductor cable portions attached at one end to said first to two or more enclosures;
said first of said one or more multi-conductor cable portions attached at a second end to said second of two more enclosures;
said first of said one or more multi-conductor cable portions operative to convey electrical energy to components from said first of said two or more enclosures to said second of said two or more enclosures;
said position-sensitive switch enclosed in said first of two or more enclosures;
said momentary switch enclosed in said first of two or more enclosures; and
said one or more embedded programs stored in said microcontroller.

5. A claim as in claim 1 further comprising:
said first of said two or more 3D accelerometers enclosed in a first of said two or more enclosures;
said battery enclosed in said first of said two or more enclosures;
said microcontroller enclosed in said second of said two or more enclosures;
said silent vibratory transducer enclosed in said first of said two or more enclosures a second of said two or more 3D accelerometers enclosed in a second of said two or more enclosures;
said necklace portion attached at a first end to said first of two or more enclosures;
said necklace portion attached at a second end to said second of two or more enclosures;
said first of said one or more multi-conductor cable portions attached at one end to said first to two or more enclosures;
said first of said one or more multi-conductor cable portions attached at a second end to said second of two more enclosures;
said first of said one or more multi-conductor cable portions operative to convey electrical energy to components from said first of said two or more enclosures to said second of said two or more enclosures;
said position-sensitive switch enclosed in said first of two or more enclosures;
said momentary switch enclosed in said first of two or more enclosures; and
said one or more embedded programs stored in said microcontroller.

6. A claims as in claim 1 further comprising:
a third of said two or more enclosures to be positioned on a user's said back area extending below said second of said two or more enclosures;
a third of said two or more 3D accelerometers enclosed in said third of said two or more enclosures;
a second of said one or more multi-conductor cable portions;
said second of said one or more multi-conductor cable portions attached at a first end to said second of two or more enclosures;
said second of said one or more multi-conductor cable portions attached at a second end to said third of said two or more enclosures; and
said second of said one or more multi-conductor cable portions provides energy to said third of said two or more 3D accelerometers and conveys said angular position data to said microcontroller.

7. A claim as in claim 1 further comprising:
said two or more 3D accelerometers are operative to provide angular position data;
said microcontroller is operative to execute at least one stored program;
said microcontroller is operative to receive said angular position data from each of said two or more 3D accelerometers;
said microcontroller is operative to generate an alert signal when a predetermined angular position difference is calculated;
said battery provides electrical energy to all electrical components;
said multi-conductor cable conveys electrical energy to said electrical components in said each of said two or more enclosures;
said multi-conductor cable conveys said angular position data from each of said two or more 3D accelerometers that are not enclosed in the same enclosure as said microcontroller; and
said multi-conductor cable conveys said alert signal from said microcontroller to said silent vibratory transducer when said silent vibratory transducer is not located in said same enclosure as said microcontroller.

8. A method of use of said system comprising:
positioning said system such that said first of said two or more enclosures is essentially centered and located on upper chest, and said second of said two or more enclosures is essentially centered and located on upper back, with necklace portion on one side of neck and multi-conductor cable on other side of said neck; and
calibrating said system while standing erect by engaging said momentary switch.

9. A claim as in claim 8 further comprising:
positioning said system such that said third of said two or more enclosures is essentially centered and located on user's back and extends below said second of two or more enclosures.
